(19) United States
(12) Patent Application Publication
(10) Pub. No.: US 2002/0016462 A1
(43) Pub. Date: Feb. 7, 2002

(54) Process for preparing basic organic nitrogen-containing compounds by reduction with lithium aluminum hydride

(76) Inventors: Johannes Scherer, Leverkusen (DE); Gunter Rauchschwalbe, Leverkusen (DE); Erich Hammerschmidt, Bergisch Gladbach (DE); Heinz-Gerd Hartges, Leverkusen (DE)

Correspondence Address: BAYER CORPORATION, PATENT DEPARTMENT, 100 BAYER ROAD, PITTSBURGH, PA, 15205, US

(21) Appl. No.: 09/912,933

(22) Filed: Jul. 25, 2001

(30) Foreign Application Priority Data
Aug. 1, 2000 (DE) 10037408.5

(51) Int. Cl. C07D 295/027; C07D 213/02; C07D 211/02; C 07D 2 9/02; C 07D 2 7/04

(52) U.S. Cl. 544/358; 546/184; 546/251; 548/452; 548/579

(57) ABSTRACT

This invention relates to a process for preparing basic organic nitrogen-containing compounds by

(a) reduction with lithium aluminum hydride,

(b) hydrolysis of the resultant mixture by addition to an aqueous solution in which substances containing α-hydroxycarboxyl and/or α-aminocarboxyl groups are dissolved, and

(c) separation of the two resultant phases at a pH of the aqueous phase of from 7 to 12.

### BACKGROUND OF THE INVENTION

The present invention relates to a process for preparing basic organic nitrogen-containing compounds by reduction with lithium aluminum hydride and subsequent decomposition of the reaction mixture with formation of a biphasic aqueous-organic system.

The basic organic nitrogen-containing compounds can be amines, pyridines, piperidines, piperazines, pyrroles, pyrrolidines, or indoles, for example.

Basic organic nitrogen-containing compounds are important intermediates for preparing crop protection agents and pharmaceutically active compounds. Thus, for example, N-benzylpyrrolopiperidine and compounds preparable therefrom, such as pyrrolopiperidine, are of interest as components of pharmaceutical compounds with antibiotic activity (see, for example, DE-A 4,234,330).

Amines, pyridines, piperidines, piperazines, pyrroles, pyrrolidines, and indoles can be prepared in a known manner, for example, by reducing compounds containing amino groups, pyridino groups, pyrrolo groups, or indole groups or by reduction of amino acids, aminoketones, pyridine-carboxylic acids, indolylcarboxylic acids, pyrrolidinones, pyrrolidinediones, piperidinones, piperidinediones, piperazinones, or piperazinediones with lithium aluminum hydride, and amines can also be prepared, for example, by reduction of corresponding amides, imides, imines, hydrazines, hydrazones, hydroxylamines, oximes, and nitriles with lithium aluminum hydride.

In such reductions with lithium aluminum hydride, adducts are formed that must be decomposed to isolate the product. Moreover, in most cases an excess of lithium aluminum hydride is used, with the result that the excess of lithium aluminum hydride also must be decomposed prior to the isolation of the product. According to the prior art, these decompositions are carried out by addition of aqueous sodium hydroxide solution, water-containing organic solvents, and/or water, which in each case gives rise to voluminous precipitates that are difficult to remove and that adsorb the basic organic nitrogen-containing compounds prepared.

Work-up of the mixture that is present after the reaction with lithium aluminum hydride by addition of acids prevents the formation of aluminum hydroxide and/or aluminate precipitates. However, this method cannot be applied if basic organic nitrogen-containing compounds are prepared, as these compounds would form salts which are soluble in the aqueous phase and their isolation by phase separation or extraction would no longer be possible.

It is also possible to add exactly the amount of water that would be required theoretically to decompose the aluminum adduct and excess lithium aluminum hydride. In this case, a fluffy precipitate of aluminum hydroxide and lithium hydroxide is formed (Lithium Aluminium Hydride Industrial Use, company publication Chemetall, Frankfurt 1993). However, under the given conditions, it is difficult to meter in an accurate amount of water.

Finally, for working up reaction mixtures obtained in reductions with lithium aluminum hydride, it has been proposed to initially add water and to re-dissolve the resulting precipitate by addition of an excess of a 20% by weight strength aqueous solution of potassium sodium tartrate (see J.A.C.S., 70, 3788 (1948)). This process, too, has a number of disadvantages:

The addition of water to the reaction mixture results in a violent reaction that may be controllable on a laboratory scale but not on a pilot-plant-scale or on an industrial scale.

Here, too, a precipitate is initially formed that has an adverse effect on the stirrability of the mixture. The precipitate is re-dissolved only later.

The reagent concentrations used are low (7.6 g of lithium aluminum hydride in 500 ml of solvent and 500 ml of a 20% by weight strength potassium sodium tartrate solution), which means that large volumes must be handled, with the associated low space-time yields.

A large excess of potassium sodium tartrate has to be employed (about 2.4 mol, or 4.8 equivalents, per mole of lithium aluminum hydride).

Thus, there is still a need for a process for preparing basic organic nitrogen-containing compounds by reduction with lithium aluminum hydride that does not have the above-mentioned disadvantages.

### SUMMARY OF THE INVENTION

This invention, accordingly, provides a process for preparing basic organic nitrogen-containing compounds comprising

(a) reducing a reducible nitrogen-containing compound with lithium aluminum hydride to form a reduced mixture,

(b) hydrolyzing the reduced mixture by addition to an aqueous solution in which substances containing α-hydroxycarboxyl and/or α-amino-carboxyl groups are dissolved, and

(c) allowing the reaction mixture to form two phases that are separated at a pH of the aqueous phase of from 7 to 12.

### DETAILED DESCRIPTION OF THE INVENTION

The process according to the invention can be used, for example, for preparing aliphatic and aromatic amines, pyridines, piperidines, piperazines, pyrroles, pyrrolidines, and indoles. These can be obtained, for example, by reducing compounds that, in addition to amino, pyridino, piperidino, piperazino, pyrrolo, pyrrolidino, and/or indole groups, contain one or more groups that can be reduced with lithium aluminum hydride, for example, oxygen-containing groups, such as carboxylate and/or keto groups. Amines can also be obtained, for example, by reducing the corresponding amides, imides, imines, hydrazones, hydroxylamines, oximes, or nitriles.

Amines that can be prepared according to the invention are, for example, those of the formula

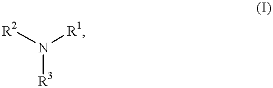

in which

R¹, R², and R³ independently of one another each represent C₁-C₈-alkyl, phenyl, benzyl or (CH)ₙX,

wherein

n represents an integer from 1 to 4, and

X represents OR⁷ or NR⁷R⁸, where R⁷ and R⁸ are hydrogen, C₁-C₈-alkyl, phenyl, or benzyl, or

R¹ and R² independently of one another each represent C₁-C₆-alkyl and

R³ represents C₄-C₂₀-alkyl, phenyl, benzyl, or ethyl.

Pyridines, piperidines, and piperazines that can be prepared according to the invention are, for example, those of the formulas

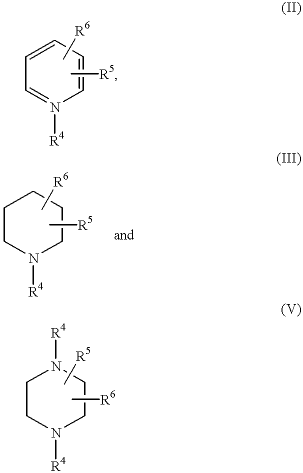

in which

R⁴, R⁵, and R⁶ independently of one another each represent hydrogen, C₁-C₈-alkyl, phenyl, benzyl, X, or (CH)ₙX,

wherein

n represents an integer from 1 to 4, and

X represents OR⁷ or NR⁷R⁸, wherein R⁷ and R⁸ are hydrogen, C₁-C₈-alkyl, phenyl, or benzyl, or

R⁴ is as defined above and R⁵ and R⁶ together form a —CH₂—NR⁹—CH₂— bridge in which R⁹ represents hydrogen, C₁-C₈-alkyl, phenyl, or benzyl.

Indoles that can be prepared according to the invention, are, for example, those of the formula

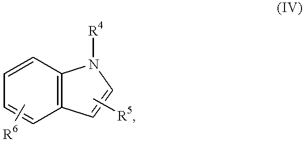

in which

R⁴, R⁵, and R⁶ each have the meanings given under formulas (II), (III), and (V), but where R⁵ and R⁶ together may not form a —CH₂—NR⁹—CH₂— bridge.

Pyrroles and pyrrolidines that can be prepared according to the invention are, for example, those of the formulas (VI) and (VII)

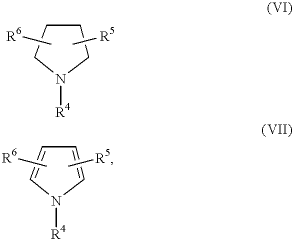

in which

R⁴, R⁵, and R⁶ each have the meanings given under formulas (II), (III), and

Preference is given to preparing compounds of the formulas (II), (III), (V), and (VI) in which

R⁴ represents hydrogen, methyl, ethyl, phenyl, benzyl, or (CH₂)nX and

R⁵ and R⁶ independently of one another each represent hydrogen, C₁-C₄-alkyl, phenyl, benzyl, X, or (CH)ₙX,

wherein

n represents 1 or 2, and

X represents OR⁷ or NR⁷R⁸, where R⁷ and R⁸ each represents hydrogen, methyl, ethyl, phenyl, or benzyl, or

R⁴ is as defined above and R⁵ and R⁶ together form a —CH₂—NR⁹—CH₂— bridge in which R⁹ represents hydrogen, methyl, ethyl, phenyl, or benzyl.

Particular preference is given to preparing compounds of the formulas (II), (III), and (VI) in which

R⁴ represents hydrogen, methyl, ethyl, phenyl, or benzyl, and

R⁵ and R6 independently of one another each represent hydrogen, C₁-C₄-alkyl, phenyl, benzyl, X, or (CH)ₙX,

wherein

n represents 1 or 2, and

X represents OH or NH₂, or

R⁴ is as defined above and R⁵ and R⁶ together form a —CH₂—NR⁹—CH₂— bridge in which R⁹ represents hydrogen, methyl, ethyl, phenyl, or benzyl.

According to the invention, particular preference is given to preparing 3-amino-1-benzylpyrrolidine or 8-benzyl-2,8-diazabicyclo[4.3.0]-nonane, also referred to as N-benzylpyrrolopiperidine or “BEPP” for short, where 3-amino-1-benzyl-pyrrolidinedione and 8-benzyl-7,9-dioxo-2,8-diazabicyclo[4.3.0]nonene or “DOPP” for short, respectively, are reduced with lithium aluminum hydride.

The reduction with lithium aluminum hydride can be carried out in a manner known per se (see, for example, Lithium Aluminium Hydride Industrial Use, company publication Chemetall, Frankfurt (1993)). The reduction is usually carried out at temperatures in the range from 5 to 60° C. in the presence of a solvent and with from 0.25 to 4 mol of lithium aluminum hydride per equivalent of the substance to be reduced. Suitable solvents are, for example, acyclic and cyclic mono- and oligoethers containing, for example, from 4 to 10 carbon atoms, such as diethyl ether, diisopropyl ether, methyl tert-butyl ether, dibutyl ether, ethylene glycol dimethyl ether, diethylene glycol dimethyl ether, tetraethylene glycol dimethyl ether, tetrahydrofuran, 2-methyltetrahydrofuran, and dioxane. The ethers can also be employed as a mixture with one another and/or in a mixture with other organic solvents that are inert to lithium aluminum hydride, for example, hydrocarbons such as benzene, toluene, xylene, and/or petroleum ether.

It is an essential feature of the present invention that the mixture present after the reduction with lithium aluminum hydride has ended is added to an aqueous solution in which substances containing α-hydroxy-carboxyl and/or α-aminocarboxyl groups are dissolved.

Suitable α-hydroxycarboxyl-group-containing substances are, for example, α-hydroxycarboxylic acids having from 1 to 6 OH groups and from 2 to 10 carbon atoms, where at least one OH group is located in a position α to a carboxyl group. The carbon skeleton of such α-hydroxy-carboxylic acids can be straight-chain or branched. Preferred α-hydroxy-carboxylic acids are those having from 1 to 5 OH groups and from 2 to 6 carbon atoms, such as glycolic acid, tartronic acid, malic acid, citric acid, tartaric acid, gluconic acid, sugar acids, mannosucceric acid, and mocic acid.

Suitable α-aminocarboxyl-group-containing substances are, for example, α-aminocarboxylic acids having from 1 to 4 amino groups and from 2 to 20 carbon atoms, where at least one primary, secondary, or tertiary amino group is located in a position α to a carboxyl group. The carbon skeleton of such α-aminocarboxylic acids can be straight-chain or branched. Preferred α-aminocarboxylic acids are those having from 1 to 3 amino groups and from 2 to 14 carbon atoms, such as glycine, alanine, aspartic acid, asparagine, glutamic acid, glutamine, nitrilotriacetic acid, ethylenediaminetetraacetic acid, diethylenetriaminepentaacetic acid, and iminodisuccinic acid.

It is also possible to use aqueous solutions in which a plurality of α-hydroxycarboxyl-group-containing substances, a plurality of α-amino-carboxyl-group-containing substances, or both α-hydroxycarboxyl-group-containing substances and α-aminocarboxyl-group-containing substances are present.

Carboxyl groups can be present in the form of free carboxylic acid radicals (i.e., COOH) or as salts, for example, as alkali metal salts (for example, COO⁻ Na⁺). Amino groups can be present in free form (i.e., —NH₂, —NHR, or —NR₂) or as ammonium salts (for example, —NH₃⁺ Cl⁻).

Hereinbelow, substances that contain α-hydroxycarboxyl groups and α-aminocarboxyl groups are together also referred to as “HAC”. One equivalent of HAC is the amount of HAC that contains one mole of α-hydraxycarboxyl groups and α-aminocarboxyl groups.

It is advantageous to use not less than 1 equivalent of HAC per mole of lithium aluminum hydride and to avoid too large an excess of HAC. The formation of undesirable precipitates during work-up of the reaction mixture from the reduction with lithium aluminum hydride can be avoided, for example, when from 1 to 3 equivalents of HAC are used. Preference is given to using from 1 to 2 equivalents of HAC.

The HAC can be employed in aqueous solutions of a concentration of, for example, from 5 to 60% by weight, preferably from 10 to 40% by weight.

The phase separation to be carried out according to the invention can be carried out, for example, at temperatures of from 5 to 100° C., preferably at from 30 to 80° C. If the temperature chosen for the phase separation is higher than the boiling point of one of the components of the mixture to be separated, the phase separation can also be carried out under superatmospheric pressure.

Prior to the phase separation, the pH of the aqueous phase is preferably from 7.5 to 9.5.

If required or desired, the pH can be adjusted after the hydrolysis to the pH desired for the phase separation by addition of alkaline inorganic substances, such as sodium hydroxide, potassium hydroxide, calcium hydroxide, sodium carbonate, or ammonia. Alkaline inorganic substances can be added neat or in the form of aqueous solutions, for example.

Furthermore, it is advantageous to stir the mixture vigorously prior to phase separation.

Following phase separation, the major part of the basic organic nitrogen-containing compound prepared according to the invention is in the upper organic phase. Further amounts of the basic organic nitrogen-containing compound can be obtained by extracting the aqueous phase after phase separation. Suitable extracting agents are, for example, the solvents and solvent mixtures described above for carrying out the reduction or other solvents that are water-immiscible or sparingly miscible with water.

Further amounts of the basic organic nitrogen-containing compound can be brought into the organic phase by adding, prior to phase separation, electrolytes having a salting-out effect. Examples of electrolytes are hydrochloric acid, sulfuric acid, phosphoric acid, nitric acid, hydrobromic acid, and alkali metal and ammonium chlorides, bromides, sulfates, hydrogen sulfates, phosphates, hydrogen phosphates, dihydrogen phosphates, and nitrates.

The compound that has been prepared is ultimately present as a solution in the solvent used for the reduction, if appropriate, additionally as an organic extract from the aqueous phase. Frequently, it is possible to use the compounds prepared directly in this form. If desired, it is also possible to remove the solvent or extracting agent, for example, by distillation, if appropriate under reduced pressure.

The process according to the invention has a number of surprising advantages:

By (if appropriately metered) addition of the mixture that is present after the reduction to an aqueous solution that contains substances with α-hydroxycarboxyl and/or α-aminocarboxyl groups, it is possible to carry out the decomposition with controlled development of heat and hydrogen, even in the case of large amounts of the mixture.

During the process, no precipitates that must be removed or dissolved are formed.

No crusts or lumps that can trap undecomposed reduction mixture are formed during the decomposition.

The phase separation takes place without formation of slime or emulsions.

The vessel in which the reduction is carried out remains moisture-free and it is therefore not necessary to dry the vessel separately in a complicated manner prior to the next batch.

The reduction can be carried out with higher reagent concentrations.

It is possible to use less α-hydroxycarboxyl- and/or α-amino-carboxyl-group-containing substance than in the different process according to J.A.C.S., 70, 3738 (1970).

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

Under nitrogen, 52.9 g lithium aluminum hydride were initially charged in a flask in a mixture of 315 g of dry tetrahydrofuran and 135 g of dry toluene. At 90° C., 169.4 g of molten DOPP were metered into this suspension from a dropping funnel heated at 110° C. Evolution of hydrogen, which initially was moderate, increased over time. The reflux temperature dropped continuously to 77° C. After the addition had ended, stirring at reflux was continued for 5 hours and the suspension was then cooled to 20° C. The low-viscosity suspension was transferred into a dry dropping funnel that had been flushed with nitrogen. In a second flask, 340.2 g of citric acid (monohydrate) were initially charged in 1079.4 g of 7.4% by weight strength sulfuric acid. At 10° C., the reaction suspension from the dropping funnel was metered into this solution in the course of one hour (evolution of hydrogen). An organic and an aqueous phase formed. Stirring was continued at 60° C. for one hour. The content of the flask was cooled to 30° C. and the pH was then adjusted to pH 8.5 by addition of 990 g of aqueous sodium hydroxide solution (15% by weight strength). The temperature was then increased to 50° C. and the mixture was stirred at this temperature for one hour. The organic phase was then separated off and the aqueous phase was extracted at 50° C. with a mixture of 280 g of tetrahydrofuran and 120 g of toluene. The organic phase which had been separated off contained 120 g of pure N-benzylpyrrolopiperidine (“BEPP”) and the extract contained 10 g of BEPP. The aqueous phase was found to contain another 0.5 g of BEPP (HPLC, external standard). The two organic phases were combined and most of the solvent was removed under reduced pressure to give 149.5 g of an oil that contained 86.5% by weight of BEPP (HPLC, external standard). This corresponds to a yield of 88.1% of theory.

### Example 2

Under nitrogen, 370 g of 2-methyltetrahydrofuran and 76 g of lithium aluminum hydride were initially charged in a flask. At 103° C., a solution of 244 g of DOPP in 163 g of 2-methyltetrahydrofuran was added to the suspension from a dropping funnel heated at 40° C. Evolution of hydrogen, which was moderate initially, increased over time, the reflux temperature falling continuously to 78° C. After the addition of DOPP had ended, stirring at reflux was continued for 3 hours and the mixture was then cooled to 20° C. The resulting suspension was transferred into a dry dropping funnel that had been flushed with nitrogen. In a second flask, 504 g of citric acid monohydrate were initially charged in 1017.6 g of 11.6% by weight strength sulfuric acid. At 10° C., the suspension from the dropping funnel was metered into the solution over the course of 1 hour. The mixture was stirred at 60° C. for another hour. The content of the flask was cooled to 30° C. and at this temperature the pH was adjusted to pH 8.5 by addition of 443 g of 15% by weight strength aqueous sodium hydroxide solution. The temperature was then increased to 60° C. and the mixture was stirred at this temperature for 1 hour. The organic phase was separated off and the aqueous phase was extracted with 533 g of 2-methyltetrahydrofuran. The organic phase contained 180 g of pure BEPP and the extract contained 10 g of BEPP. No more BEPP was detected in the aqueous phase. The two organic phases were combined and most of the solvent was removed under reduced pressure to give 217.2 g of an oil having a content of 89.1% by weight of BEPP (HPLC, external standard).

This corresponds to a yield of 89.5% of theory.

### Example 3

Under nitrogen, 21.9 g of lithium aluminum hydride were initially charged in a flask in a mixture of 210 g of dry tetrahydrofuran and 90 g of dry toluene. At 90° C., 58.8 g of 3-amino-1-benzylpyrrolidinedione (96.2%), dissolved in a mixture of 70 g of dry tetrahydrofuran and 30 g of dry toluene, were metered into the gray suspension from a dropping funnel. Evolution of H₂, which was moderate initially, increased over time. The reflux temperature fell continuously to 76° C. After the addition had ended, stirring at reflux was continued for 5 hours and the suspension was then cooled to 20° C. The low-viscosity suspension was transferred into a dry dropping funnel that had been flushed with nitrogen. In a second flask, 145.2 g of citric acid (monohydrate) and 33.9 g of sulfuric acid (100% strength) were initially charged in 600.0 g of water. At 10° C., the reaction suspension from the dropping funnel was metered into this solution over the course of 1 hour (evolution of H₂). An organic and an aqueous phase were formed. Stirring at 60° C. was continued for 1 hour. The content of the flask was cooled to 30° C. (pH 2.1), and at this temperature the pH was adjusted to pH 8.5 by addition of 360.0 g of aqueous sodium hydroxide solution (15% strength). The temperature was then increased to 50° C., and the mixture was stirred at this temperature for 1 hour. The organic phase was separated off and the aqueous phase was re-extracted twice at 50° C. using a mixture of 140 g of tetrahydrofuran and 60 g of toluene. The organic phases were combined and most of the solvent was removed under reduced pressure to give 41.5 g of an oil which had a content of 86.7% (GC) of 3-amino-1-benzylpyrrolidine (73.4% of theory).

What is claimed is:

1. A process for preparing basic organic nitrogen-containing compounds comprising
(a) reducing a reducible nitrogen-containing compound with lithium aluminum hydride to form a reduced mixture,
(b) hydrolyzing the reduced mixture by addition to an aqueous solution in which substances containing α-hydroxycarboxyl and/or α-amino-carboxyl groups are dissolved, and
(c) allowing the reaction mixture to form two phases that are separated at a pH of the aqueous phase of from 7 to 12.

2. A process according to claim 1 wherein an amine, a pyridine, a piperidine, a piperazine, a pyrrole, a pyrrolidine, or an indole is prepared by reducing a compound that contain one or more amino, pyridino, piperidino, piperazino, pyrrolo, pyrrolidino, or indole groups, and further contain one or more groups that can be reduced with lithium aluminum hydride.

3. A process according to claim 1 wherein an amine is prepared by reducing a corresponding amide, imide, imine, hydrazone, hydroxylamine, oxime, or nitrile.

4. A process according to claim 1 wherein 3-amino-1-benzyl-pyrrolidine is prepared from 3-amino-1-benzyl-pyrrolidinedione or 8-benzyl-2,8-diazabicyclo[4.3.0]nonane is prepared from 8-benzyl-7,9-dioxo-2,8-diazabicyclo[4.3.0.]nonane.

5. A process according to claim 1 wherein the substance containing α-hydroxycarboxyl groups is an α-hydroxycarboxylic acid having 1 to 6 OH groups and 2 to 10 carbon atoms, wherein at least one OH group is located in a position α to a carboxyl group.

6. A process according to claim 1 wherein the substance containing α-aminocarboxyl groups is an α-aminocarboxylic acid having 1 to 4 amino groups and 2 to 20 carbon atoms, wherein at least one primary, secondary, or tertiary amino group is located in a position a to a carboxyl group.

7. A process according to claim 1 wherein from 1 to 3 equivalents of the substances containing α-hydroxycarboxyl groups and/or α-aminocarboxyl groups are employed per mole of lithium aluminum hydride.

8. A process according to claim 1 wherein the substances containing α-hydroxycarboxyl groups and/or α-aminocarboxyl groups are used in aqueous solutions of a concentration of from 5 to 60% by weight.

9. A process according to claim 1 wherein the phase separation is carried out at a temperature of from 5 to 100° C.

10. A process according to claim 1 wherein the pH of the aqueous phase prior to phase separation is from 7.5 to 9.5.

11. A process according to claim 1 wherein the aqueous phase that remains after removal of the organic phase is extracted to obtain further amounts of the basic organic nitrogen-containing compound.
